(19) United States
(12) Patent Application Publication
(10) Pub. No.: US 2004/0166492 A1
(43) Pub. Date: Aug. 26, 2004

(54) Quantitative detection of dekkera and saccharomyces

(76) Inventors: Stacia R. Engel, Modesto, CA (US); Nancy A. Irelan, Modesto, CA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/358,929

(22) Filed: Feb. 5, 2003

Related U.S. Application Data
(60) Provisional application No. 60/354,768, filed on Feb. 5, 2002.

(51) Int. Cl. C12Q 1/68; C07H 21/04

(52) U.S. Cl. 435/6; 536/24.1

(57) ABSTRACT

DNA sequences are provided which are useful as hybridization probes for the real-time quantitative detection of Dekkera and Saccharomyces species.

### FIELD OF THE INVENTION

This application claims benefit of U.S. provisional application Serial No. 60/354,768, filed Feb. 5, 2002 and relates to the use of probes in TaqMan® quantitative PCR assays for the detection of Dekkera bruxellensis (anamorph Brettanomyces bruxellensis), Saccharomyces cerevisiae, and S. bayanus. The use of these assays allows the detection of specific beverage-related yeasts and their quantification on plant tissue, in juice, and in wine.

### BACKGROUND OF THE INVENTION

The alcoholic fermentation of grape juice (must) to produce wine is a complex biological process in which yeasts, most typically Saccharomyces cerevisiae and S. bayanus, convert sugars to ethanol, water, and carbon dioxide. There are many different strains of S. cerevisiae and S. bayanus, some of which differ widely with respect to their metabolic processes and by products produced. In fact, while Saccharomyces yeasts are predominantly responsible for the fermentation, some strains are also capable of spoiling the final product. Another widespread yeast species notorious for causing wine spoilage is Dekkera bruxellensis (anamorph Brettanomyces bruxellensis), which commonly produces medicinal, “Band-Aid”, horse sweat, or barnyard off-notes, among others (Licker et al., 1999, Am. Chem. Soc. Symp. Ser. 714:96-115). In the wine industry, D. bruxellensis is commonly referred to as simply “Brett.”

Past techniques used for identifying fermentation-related microorganisms, especially yeast, have included evaluations of the following: colony morphology, fermentation performance, tolerance to various stresses (e.g., ethanol, heat, etc.), phenotypes with functional relevance (e.g., flocculation), nutritional requirements (e.g., oxygen, specific sugars, etc.), and resistance/sensitivity to specific anti-microbial agents, (e.g., cycloheximide, SO₂, etc.). These methods, however, bear numerous disadvantages such as lengthy analysis times, inadequate resolution, ambiguous results, or lack of reproducibility. Of interest to the present invention are the disclosures of Rodrigues et al. (2001, J. Appl. Micro., 90:588-99) which relate to the detection of Dekkera/Brettanomyces using a selective medium.

Early developments in molecular biology and protein chemistry have provided other methods for identifying microorganisms, including DNA restriction fragment length polymorphisms (RFLPs), protein electrophoresis patterns (allozymes), and chromosome fingerprinting (karyotyping). Such techniques have been applied to the identification of fermentation-related microorganisms as recorded in the following: Casey et al., 1990, J. Am. Soc. Brew. Chem. 48(3):100-6; Degre et al., 1989, AJEV40(4):309-15; Guillamon et al., 1992, Syst. Appl. Micro. 19:122-32; Hoeben et al., 1986, Curr. Genet. 10:371-9; Mozina et al., 1997, Lett. Appl. Micro. 24(4):311-5; Paffetti et al., 1995, Res. Micro. 146:587-94; Panchal et al., 1987, J Inst. Brew. 93:325-7; Querol et al., 1992, Syst. Appl. Micro. 15:439-46; Vezinhet et al., 1990, Appl. Micro. Biotech. 32:568-71; and Vezinhet et al., 1992, AJEV, 43(1):83-6. Of interest to the present invention are the disclosures of Kuniyuki et al. (1984, AJEV35(3):143-5) and Munnoch (1988, Dissertation Vol. 49-11B, University of Reading, UK), both of which are directed to enzyme-linked immunosorbent assays (ELISA) for the detection of Brettanomyces contaminants in wine production.

Polymerase chain reaction (PCR)-based techniques have also been used to detect specific microorganisms. In a typical PCR assay, oligonucleotide primers which are complementary to the 5′ and 3′ ends of a specific DNA sequence are used to amplify the target sequence, thereby allowing detection. The DNA sample of interest is first heat-denatured, and the specific primers are annealed to the sample DNA, and then extended in the presence of nucleotide triphosphates and a DNA polymerase such as Taq polymerase. Repeated thermal cycling to denature the template DNA and anneal/extend the specific primers results in amplification of the target DNA sequence. For examples of the application of PCR for the detection of fermentation-related microorganisms see the following: de Barros Lopes et al., 1996, Appl. Environ. Microbiol. 62(12):4514-40; Fell, 1993, Molec. Marine Biol. Biotech., 2(3):174-180; Fell, 1995, J. Inst. Micro. 14(6):475-477; Lavallee et al., 1994, AJEV 45(1):86-91; Lieckfeldt et al., 1993, J. Basic Micro. 33(6):413-425; and Ness et al., 1993, J Sci. Food Agric. 62:89-94. Of interest to the present invention are the disclosures of Ibeas et al. (1996, Appl. Environ. Microbiol. 62(3):998-1003) which relate to the detection of Dekkera-Brettanomyces strains in sherry by a nested PCR method. Also of interest are the disclosures of Stender et al. (2001, Appl. Environ. Microbiol. 67(2):938-41) which are directed to the detection of D. bruxellensis using peptide nucleic acid (PNA) probes.

In S. cerevisiae, the 18S, 5.8S, 28S, and 5S nuclear ribosomal RNA (rRNA) genes are organized in operons tandemly-repeated approximately 150 times (Wai et al., 2000, Nuc. Acids Res. 28(18):3524-34). This high copy number makes rRNA operons extremely attractive targets for the design and application of molecular probes. The internal transcribed spacer (ITS) region lies between the 18S and 28S rRNA genes and contains two variable non-coding spacers (often referred to as ITS1 and ITS2), as well as the 5.8S rRNA gene (White et al., 1990, in PCR Protocols, eds. Innes et al., pp. 315-22). Sequence analyses of the ITS region have revealed differing levels of intra- and interspecies variation for a number of fungi (DeScenzo et al., 1999, Phytopath. 89:884-93; Magee et al., 1987, J. Bacteriol. 169:1639-43; O'Donnell et al., 1992, Curr. Genet. 22:213-20; Oda et al., 2001, Biosci. Biotechnol. Biochem. 65(1):164-6; and Sugita et al., 2000, Microbiol. Immunol. 44(6)455-61), making the ITS region well-suited for use in the design of species-specific detection and identification methods.

Kumeda et al. (1996, Appl. Environ. Microbiol. 62(8):2947-52) describe the use of PCR to amplify the ITS region in order to differentiate species of Aspergillus Section Flavi. The ITS region was amplified using universal primers, with the PCR product analyzed by the principle of single-strand conformation polymorphism (SSCP). Gardes et al. (1996, in Species Diagnostics Protocols: PCR and Other Nucleic Acid Methods, ed. J. P. Clapp, pp. 177-86) describe RFLP analysis of fungal ITS regions amplified by PCR. The PCR amplification of fungal ITS regions has also been described using other than universal primers. That is, PCR primers directed to unique sequences in the ITS 1 and/or ITS2 regions of fungal pathogens allow for more specificity in identifying certain fungi. Gardes and Bruns (1993, Mol. Ecol. 2:113-8) identified ITS primers which allow differentiation of DNA from basidiomycetes against ascomycetes. See also Hamelin et al., 1996, Appl. Environ. Microbiol. 62(11):4026-31; Mazzola et al., 1996, Phytopath. 86(4):354-60; O'Gorman et al., 1994, Can. J Botany 72:342-346; and Ligon et al., 1996, U.S. Pat. No. 5,585,238.

Of interest to the present invention are the disclosures of U.S. Pat. Nos. 6,248,519 and 6,287,779 directed to oligonucleotide primers specific for the ITS regions of fermentation-related microorganisms such as Botrytis, Penicillium, Brettanomyces/Dekkera, Candida, Debaryomyces, Hanseniaspora, Kluyveromyces, Metschnikowia, Pichia, Saccharomyces, Saccharomycodes, Torulaspora, and Zygosaccharomyces, the disclosures of which are hereby incorporated by reference. Also of interest to the present invention is the disclosure of co-owned copending U.S. patent application Ser. No. 09/710,753 filed Nov. 11, 2000, and hereby incorporated by reference, which is directed to improved methods for rapid isolation and amplification of nucleic acids present in beverages such as fruit juices and wine.

The use of 5′ nuclease assays in combination with dual-labeled fluorogenic probes, as in TaqMan® assays, can be performed with commercially-available equipment, such as the ABI PRISM® 5700 and 7700 Sequence Detection Systems (Applied Biosystems, Foster City, Calif.), and allow for the quantitative detection of target DNAs. The TaqMan® assay consists of PCR in which a specific oligonucleotide probe, designed to anneal to the target DNA between the 5′ and 3′ primers, is used in conjunction with the primers to detect the presence of, and quantify, the target DNA. The dual-labeled probe contains fluorescent dyes on both ends, one of which acts as a reporter, the other as a quencher. The proximity of the reporter at the 5′ end and the quencher at the 3′ end results in suppression of the fluorescence. When the probe anneals to the target DNA, the 5′ nuclease activity of the DNA polymerase, which is extending the PCR primers, cleaves the reporter dye from the 5′ end of the probe, freeing it from the influence of the quencher and resulting in fluorescence emission. Since the cleavage of the reporter dye destroys the probe for further use, accumulation of PCR product results in an increase in fluorescence output, allowing quantification of the target DNA (Heid at al., 1996, Genome Res. 6:986-94).

Dual-labeled fluorogenic probes have been used, such as in TaqMan® assays, to detect plant pathogenic bacteria (Schaad et al., 1999, Plant Dis. 83:1095-100), plant pathogenic viruses (Schoen et al., 1996, Phytopath. 86:993-9), plant pathogenic fungi (Beck and Barnett, 2001, U.S. Pat. No. 6,319,673; and Frederick et al., 2001, U.S. Pat. No. 6,316,195), and human pathogenic fungi (Brandt et al., 1998, J. Clin. Micro. 36:2057-62; and Shin et al., 1999, J. Clin. Micro. 37:165-70). Dual-labeled fluorogenic probes and TaqMan® assays have not been used hereto for the detection and quantification of wine-related microorganisms, especially potential spoilage yeasts. Nevertheless, there remains a desire in the art for improved methods for the early detection, and prevention, of potential spoilage issues in wine.

### SUMMARY OF THE INVENTION

The present invention is directed to methods for the identification and quantification of the following food- and beverage-related yeast species: Dekkera bruxellensis (anamorph Brettanomyces bruxellensis), Saccharomyces cerevisiae, and S. bayanus. Specifically, the invention provides nucleic acid hybridization probes, including dual-labeled fluorogenic probes, useful in TaqMan® quantitative polymerase chain reaction (PCR) assays. In particular, oligonucleotide probes are provided which consist of a member selected from the group consisting of SEQ ID NOS 1 and 2 and their complementary sequences. It will be apparent to those of ordinary skill in the art that, while the probes of SEQ ID NOS 1 and 2 are particularly preferred for the detection of D. bruxellensis, S. cerevisiae, and S. bayanus, shorter and longer probes comprising portions of SEQ ID NOS 1 and 2, and corresponding contiguous nucleotides within SEQ ID NOS 3 and 4, respectively (and their complementary sequences), will also be useful in the qualitative and quantitative detection of D. bruxellensis, S. cerevisiae, and S. bayanus. Thus, the invention provides oligonucleotide probes wherein the probe or its complement specifically binds to at least a portion of the ITS region of SEQ ID NOS: 3, 4 or 5 or which specifically binds to at least a portion of the ITS region of a fermentation-related organism selected from the group consisting of D. bruxellensis, S. bayanus, and S. cerevisiae wherein the probe or its complement is selected from the group consisting of probes which contain at least 20 contiguous nucleotide bases from one of SEQ ID NOS: 1 or 2, probes which contain at least 20 contiguous nucleotide bases from one of SEQ ID NOS: 1 or 2 contiguous with 1 to 15 flanking nucleotide bases in the 5′ and/or 3′ direction of SEQ ID NOS: 3 to 5, and probes of 20 nucleolide bases or longer which contain at least 5 contiguous nucleotide bases from one of SEQ ID NOS: 1 or 2 continuous with from 1 to 15 flanking nucleotide bases in the 5′ and/or 3′ direction of SEQ ID NOS: 3 to 5.

While the oligonucleotide probes of the invention may also be used in more conventional PCR or hybridization assays, they are preferably labeled with a fluorescent reporter dye at the 5′ end and a corresponding fluorescent quencher dye at the 3′ end for use in real-time quantitative PCR.

The invention thus provides methods for the detection of D. bruxellensis, S. cerevisiae, and S. bayanus in a sample comprising the steps of isolating DNA from the sample and detecting the isolated DNA by hybridization with the oligonucleotide probe. The invention also provides methods for the quantitative detection of D. bruxellensis, S. cerevisiae, and S. bayanus in a sample comprising the following steps: (a) isolating DNA from the sample; (b) subjecting said DNA to polymerase chain reaction amplification using a pair of oligonucleotide primers and the dual-labeled fluorogenic probe according to the invention; and (c) monitoring said PCR amplification by detecting the fluorescence produced by the dual-labeled fluorogenic probe.

The invention also provides kits for the detection and quantitative detection of D. bruxellensis, S. cerevisiae, and S. bayanus in a sample comprising a pair of primers capable of amplifying portions of the internal transcribed sequence in those organisms and an oligonucleotide probe selected from either SEQ ID NOS 1 or 2.

### BRIEF DESCRIPTION OF THE SEQUENCES IN THE SEQUENCE LISTING

SEQ ID NO 1: Oligonucleotide sequence of Dekkera bruxellensis probe BRI;

SEQ ID NO 2: Oligonucleotide sequence of Saccharomyces cerevisiae/bayanus probe SX1;

SEQ ID NO 3: Internal transcribed spacer sequence of Dekkera bruxellensis;

SEQ ID NO 4: Internal transcribed spacer sequence of Saccharomyces cerevisiae;

SEQ ID NO 5: Internal transcribed spacer sequence of Saccharomyces bayanus;

SEQ ID NO 6: Oligonucleotide sequence ITS5;

SEQ ID NO 7: Oligonucleotide sequence ITS4.

### DETAILED DESCRIPTION OF THE INVENTION

The present invention provides unique DNA sequences which are useful for the identification and/or quantitative detection of certain fermentation-related microorganisms. Practice of the invention is not limited to detecting the presence of these microorganisms in fermentation-related operations, however, as the DNA sequences of the present invention can be used to detect the presence of such microorganisms from any source. The unique DNA sequences of the present invention can be used as probes in quantitative polymerase chain reaction (PCR) analyses, or other hybridization assays, for the detection, identification, and quantification of fermentation-related microorganisms. The DNA sequences of the present invention include detection probes derived from the internal transcribed spacer (ITS) region of the ribosomal RNA (rRNA) operon, and may be used in conjunction with universal primers such as ITS5 (SEQ ID NO 6) and ITS4 (SEQ ID NO 7) which are generally capable of amplifying all fungal species in molecular amplification assays. The interspecies variability in the rRNA operon makes it a useful target for the development of species-specific detection markers. The ITS sequences of different species can be aligned and compared to one another, and species-specific primers and/or probes designed in areas unique to certain species. Additional primers to amplify the entire ITS region can be synthesized according to White et al. (1990, in PCR Protocols, eds. Innes et al., pp. 315-22), the contents of which are hereby incorporated by reference). Methods for use of the primer sequences in PCR analysis are well known in the art as demonstrated by the following patents, the contents of all of which are hereby incorporated by reference: U.S. Pat. Nos. 4,683,195; 4,683,202; 5,585,238; 6,080,543; 6,248,519; and 6,287,779.

The present invention is directed to methods for the detection, identification, and quantification of yeasts using dual-labeled fluorogenic nucleic acid probes in conjunction with PCR according to the methods of Heid et al. (1996, Genome Res. 6:986-94). In particular, the present invention is directed to the detection, identification, and quantification of Dekkera and Saccharomyces yeasts, more particularly D. bruxellensis (anamorph Brettanomyces bruxellensis), S. cerevisiae, and S. bayanus. According to one method, a sample potentially containing one or more species of yeast is subjected to PCR analysis, under appropriate thermal cycling conditions, to which has been added the particular species-specific dual-labeled fluorogenic hybridization probe. The dual-labeled probe will hybridize to the target sequence of the yeast, if present in the sample, and the 5′ nuclease activity of the Taq polymerase will cleave the fluorescent dye reporter from the 5′ end of the probe during the extension phase of PCR. The cleavage of the 5′ reporter from the probe releases it from the quenching influence produced by the second fluorescent dye molecule attached to the 3′ end of the probe, thereby allowing detection. Because each probe molecule is rendered inactive after the initial cleavage of the 5′ reporter, computerized fluorescence detection after each thermal cycle allows for the additive quantification of the target in real time.

The present invention also relates to the preparation of “kits” containing elements for detecting fermentation-related microorganisms. Such a kit may comprise a carrier to receive therein one or more containers, such as tubes or vials. Unlabeled or detectably-labeled oligonucleotide primers may be contained in one or more of the containers. Dual-labeled fluorogenic probes may also be contained in one or more of the containers. The oligonucleotide primers may be present in lyophilized form, or in an appropriate buffer. One or more enzymes or reagents for use in PCR may be contained in one or more of the containers. The enzymes or reagents may be present alone or in admixture, and in lyophilized form or in appropriate buffers. The kit may also contain any other component necessary for carrying out the present invention, such as buffers, extraction agents, enzymes, pipettes, pipette tips, plates, nucleic acids, nucleoside triphosphates, nucleotide triphosphates, filter paper, gel materials, transfer materials, and autoradiography supplies.

### EXAMPLES

### Example 1

### Culture of Yeast and Fungal Isolates

Viable isolates of Dekkera bruxellensis, Saccharomyces cerevisiae, S. bayanus, Candida krusei, C. parapsilosis, C. stellata, Debaryomyces carsonii, Hanseniaspora guilliermondii, Kluyveromyces thermotolerans, Metschnikowia pulcherrima, Pichia anomala, P. kluyveri, Saccharomycodes ludwigii, Torulaspora delbrueckii, Zygosaccharomyces bailii, and Z. bisporus were obtained from E. & J. Gallo Winery, American Type Culture Collection, or Centraalbureau voor Schimmelcultures (Table 1). Yeasts were grown on any of several media of choice.

TABLE 1
Test Isolates

| Species Name | ID Number | Source |
|---|---|---|
| Dekkera bruxellensis | ATCC10560 | ATCC¹ |
| Dekkera bruxellensis | Y153 | Gallo of Sonoma² |
| Dekkera bruxellensis | Y207 | Gallo of Sonoma |
| Dekkera intermedia | ATCC34448 | ATCC |
| Dekkera anomala | ATCC10559 | ATCC |
| Saccharomyces cerevisiae | ATCC4127 | ATCC |
| Saccharomyces cerevisiae | GS061 | Gallo of Sonoma |
| Saccharomyces cerevisiae | GS117 | Gallo of Sonoma |
| Saceharomyces bayanus | ATCC13056 | ATCC |
| Candida krusei | GS076 | Gallo of Sonoma |
| Candida krusei | GS096 | Gallo of Sonoma |
| Candida parapsilosis | QA45 | E. & J. Gallo Winery³ |
| Candida stellata | CBS157 | CBS⁴ |
| Candida stellata | CBS2649 | CBS |
| Debaryomyces carsonii | ATCC24214 | ATCC |
| Debaryomyces carsonii | Y448 | Gallo of Sonoma |
| Hanseniaspora guilliermondii | ATCC66166 | ATCC |
| Hanseniaspora guilliermondii | GS014 | Gallo of Sonoma |
| Kluyveromyces thermotolerans | GS104 | Gallo of Sonoma |
| Kluyveromyces thermotolerans | GS138 | Gallo of Sonoma |
| Metschnikowia pulcherrima | GS011 | Gallo of Sonoma |
| Metschnikowia pulcherrima | GS066 | Gallo of Sonoma |
| Pichia anomala | ATCC34080 | ATCC |
| Pichia anomala | GS085 | Gallo of Sonoma |
| Pichia kluyveri | ATCC64303 | ATCC |
| Pichia kluyveri | GS070 | Gallo of Sonoma |
| Saccharomycodes ludwigii | ATCC34085 | ATCC |
| Torulaspora delbrueckii | GS038 | Gallo of Sonoma |
| Torulaspora delbrueckii | NS1-16 | Gallo of Sonoma |
| Zygosaccharomyces bailii | QA17 | E. & J. Gallo Winery |
| Zygosaccharomyces bailii | QA48 | E. & J. Gallo Winery |
| Zygosaccharomyces bisporus | Y476 | Gallo of Sonoma |

¹American Type Culture Collection, Rockville, MD, USA
²Gallo of Sonoma Winery, Healdsburg, CA, USA
³E. & J. Gallo Winery, Modesto, CA, USA
⁴Centraalbureau voor Schimmelcultures, The Netherlands

### Example 2

### Selection of Species-Specific Probes

Sequences presented in U.S. Pat. Nos. 6,248,519 (SEQ ID NOS 1-12 in that patent) and 6,287,779 (SEQ ID NOS 1-5 in that patent) were aligned, and probes were designed using Primer Express 1.0 (Applied Biosystems, Foster City, Calif.) in regions of maximum sequence divergence between species (Table 2).

### Example 3

### Synthesis of Species-Specific Probes

Dual-labeled fluorogenic probes having the sequences 5′-FAM-ATT ACA GGA TGC TGG GCA TAA GCC CG-TAMRA-3′ (BRI), (SEQ ID NO 1) and 5′-FAM-TGA GAG CTT TTA CTG GGC AAG AAG ACA AGA G-TAMRA-3′ (SX1), (SEQ ID NO 2) were synthesized commercially by the ABI PRISM®Primers & TaqMan® Probes Synthesis Service at Applied Biosystems (Foster City, Calif.).

TABLE 2
Species-specific Probes

| Target Species | Probe Name | Probe Sequence |
|---|---|---|
| Dekkera | BR1 | 5′-FAM-ATT ACA GGA TGC TGG |
| bruxellensis | | GCA TAA GCC CG-TAMRA-3′ (SEQ |
| | | ID NO 1) |
| Saccharomyces | SX1 | 5′-FAM-TGA GAG CTT TTA CTG GGC |
| bayanus | | AAG AAG ACA AGA G-TAMRA-3′ |
| | | (SEQ ID NO 2) |
| Saccharomyces | SX1 | 5′-FAM-TGA GAG CTT TTA CTG GGC |
| cerevisiae | | AAG AAG ACA AGA G-TAMRA-3′ |
| | | (SEQ ID NO 2) |

### Example 4

Use of Dual-labeled Fluorogenic Probes in Real-time Quantitative PCR

According to this example, DNA is extracted from a sample suspected of containing members of a Dekkera or Saccharomyces species according to conventional methods. The DNA is then subjected to real-time quantitative PCR using TaqMan® Universal PCR Master Mix (P/N 4304437, Applied Biosystems, Foster City, Calif.) in the presence of universal primers, such as ITS5 (SEQ ID NO 6) and ITS4 (SEQ ID NO 7) (White et al., 1990, in PCR Protocols, eds. Innes et al., pp. 315-22), with a dual-labeled fluorogenic probe according to the invention (SEQ ID NOS 1-2). Analysis is performed using the ABI PRISM® 5700 or 7700 Sequence Detection Systems (Applied Biosystems, Foster City, Calif.) according to the procedures set out for its use by the manufacturer.

### Example 5

### Specificity Verification of Probes to Target Species

Each probe produced according to Example 3 was screened across a panel of DNAs from different beverage-related yeasts (Table 1) to confirm its specificity to the species for which it was designed. Analyses were performed as described in Example 4, and each probe was found to detect only the target species.

### Example 6

### Use of Dual-labeled Fluorogenic Probe Sequences as Conventional Hybridization Probes

Each probe of the invention may also be used as a conventional hybridization probe according to conventional methods known to the art.

While the invention has been described in detail and with reference to specific embodiments thereof, it will be apparent to one skilled in the art that various changes and modifications can be made therein without departing from the spirit and scope thereof.

What is claimed is:

1. An oligonucleotide probe for identification of a fermentation-related microorganism, wherein said probe or its complement specifically binds to at least a portion of the ITS region of SEQ ID NOS: 3, 4 or 5 or which specifically binds to at least a portion of the ITS region of a fermentation-related organism selected from the group consisting of Dekkera bruxellensis, Saccharomyces bayanus, and Saccharomyces cerevisiae wherein said probe or its complement is selected from the group consisting of probes which contain at least 20 contiguous nucleotide bases from one of SEQ ID NOS: 1 or 2, probes which contain at least 20 contiguous nucleotide bases from one of SEQ ID NOS: 1 or 2 contiguous with 1 to 15 flanking nucleotide bases in the 5′ and/or 3′ direction of SEQ ID NOS: 3 to 5, and probes of 20 nucleotide bases or longer which contain at least 5 contiguous nucleotide bases from one of SEQ ID NOS: 1 or 2 continuous with from 1 to 15 flanking nucleotide bases in the 5′ and/or 3′ direction of SEQ ID NOS: 3 to 5.

2. The oligonucleotide probe according to claim 1 which consists of a member selected from the group consisting of SEQ ID NOS 1 and 2 and their complementary sequences.

3. The oligonucleotide probe of claim 1 having a fluorogenic reporter dye at its 5′ end and a quencher dye at its 3′ end.

4. A method for detection of Dekkera bruxellensis in a sample comprising the steps of isolating DNA from the sample and detecting the amplified DNA to by hybridization with an oligonucleotide probe of claim 1.

5. The method of claim 4 further comprising the step of subjecting said DNA to polymerase chain reaction amplification using a pair of oligonucleotide primers prior to hybridization with an oligonucleotide probe of claim 3.

6. The method of claim 5 wherein the oligonucleotide primers amplify the internal transcribed spacer sequence of Dekkera bruxellensis.

7. A method for quantitative detection of Dekkera bruxellensis in a sample comprising:
(a) isolating DNA from the sample;
(b) subjecting said DNA to polymerase chain reaction amplification using a pair of oligonucleotide primers and the dual fluorogenic probe according to claim 3;
(c) monitoring said polymerase chain reaction amplification by detecting the fluorescence produced by the dual fluorogenic probe.

8. A method for detection of Saccharomyces cerevisiae or S. bayanus in a sample comprising the steps of isolating DNA from the sample and detecting the amplified DNA to by hybridization with an oligonucleotide probe of claim 1.

9. The method of claim 8 further comprising the step of subjecting said DNA to polymerase chain reaction amplification using a pair of oligonucleotide primers prior to hybridization with an oligonucleotide probe of claim 1.

10. The method of claim 8 wherein the oligonucleotide primers amplify the internal transcribed spacer sequence of Saccharomyces cerevisiae or S. bayanus.

11. A method for quantitative detection of Saccharomyces cerevisiae or S. bayanus in a sample comprising:
(a) isolating DNA from the sample;
(b) subjecting said DNA to polymerase chain reaction amplification using a pair of oligonucleotide primers and the dual fluorogenic probe according to claim 3;
(c) monitoring said polymerase chain reaction amplification by detecting the fluorescence produced by the dual fluorogenic probe.

12. A kit comprising a carrier to receive therein one or more containers, at least one of said containers comprising an oligonucleotide probe according to claim 1.


SEQUENCE LISTING (7 sequences)

SEQ ID NO: 1 - DNA, 26 bp, Dekkera bruxellensis
attacaggatgctgggcataagcccg

SEQ ID NO: 2 - DNA, 31 bp, Saccharomyces cerevisiae/bayanus
tgagagcttttactgggcaagaagacaagag

SEQ ID NO: 3 - DNA, 377 bp, Dekkera bruxellensis
caggatgctgggcataagcccgtgcagacacgtggataagcaaggataaaaatacattaaatttatttagtttagtcaagaaagaattttaaaactttcaacaatggatctcttggttctcgcgtcgatgaagagcgcagcgaattgcgatacttaatgtgaattgcagattttcgtgaatcatcgagttcttgaacgcacattgcgccctctggtattccggagggcatgcctgtttgagcgtcatttccttctcactatttagtggttatgagattacacgagggtgttttcttcaaagggaagaggggagtgaggggataatgatttaaggtttcggccgttcattattttttcttctcccccagttatcaagt

SEQ ID NO: 4 - DNA, 754 bp, Saccharomyces cerevisiae
aagaaatttaataattttgaaaatggatttttttgttttggcaagagcatgagagcttttactgggcaagaagacaagagatggagagtccacccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatctattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgtttttttttccaaagagaggtttctctgcgtgcttgaggtataatgcaagtacggtcgttttaggttttaccaactgcggctaatctttttttatactgagcgtattggaacgttatcgataagaagagagcgtctaggcgaacaatgttcttaaagt

SEQ ID NO: 5 - DNA, 754 bp, Saccharomyces bayanus
aagaaatttaataattttgaaaatggatttttttgttttggcaagagcatgagagcttttactgggcaagaagacaagagatggagagtccagccgggcctgcgcttaagtgcgcggtcttgctaggctttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatctattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgttttttttccaaagagaggtttctctgcgtgcttgaggtataatgcaagtacggtcgttttaggttttaccaactgcggctaatctttttttatactgagcgtattggaacgttatcgataagaagagagcgtctaggcgaacaatgttcttaaagt

SEQ ID NO: 6 - DNA, 22 bp, Artificial sequence
  Oligonucleotide sequence ITS5
ggaagtaaaagtcgtaacaagg

SEQ ID NO: 7 - DNA, 20 bp, Artificial sequence
  Oligonucleotide sequence ITS4
tcctccgcttattgatatgc